(19) United States
(12) Patent Application Publication
(10) Pub. No.: US 2024/0084239 A1
(43) Pub. Date: Mar. 14, 2024

(54) COUNTING ASSEMBLY, COUNTING DEVICE AND APPLICATIONS THEREOF

(71) Applicant: SHANGHAI RUIYU BIOTECH CO., LTD., Shanghai (CN)

(72) Inventor: Kai CHEN, Shanghai (CN)

(73) Assignee: SHANGHAI RUIYU BIOTECH CO., LTD., Shanghai (CN)

(21) Appl. No.: 18/518,934

(22) Filed: Nov. 24, 2023

Related U.S. Application Data
(63) Continuation of application No. PCT/CN2022/095775, filed on May 27, 2022.

(30) Foreign Application Priority Data
May 28, 2021 (CN) 202121186301.X

(51) Int. Cl. C12M 1/34

(52) U.S. Cl. CPC C12M 41/36

(57) ABSTRACT

Embodiments of the present disclosure may teach a counting assembly, a counting device, and applications thereof. The counting assembly may include a main body. The main body may be provided with a concave sample tank, a bottom end of a sample tank being plane. A cross-sectional area of the sample tank may gradually increase from the bottom end to a top end of the sample tank. By using the counting assembly, obtaining a count of particles or cells, etc. of the sample in the sample tank may be realized by imaging the sample for one or more times, which makes the process fast and efficient, as well as minimizing the counting error generated by an uneven distribution of the particles or cells and/or a limited sampling of the particles or cells, etc.

### CROSS-REFERENCE TO RELATED APPLICATIONS

This application is a Continuation of International Application No. PCT/CN2022/095775, filed on May 27, 2022, which claims priority of Chinese Patent Application No. 202121186301.X, filed on May 28, 2021, the entire contents of which is incorporated herein by reference.

### TECHNICAL FIELD

The present disclosure relates to the technical field of a biomedical instrument, and in particular to a counting assembly, a counting device, and applications thereof.

### BACKGROUND

A counting plate is a commonly used consumable for recognizing and counting micro-particles and cells. The counting plate is widely used in the fields of life sciences, biomedicine, and medical testing, etc. At present, most of the counting plates used in a cell counter domestically and abroad are realized by referring to the design principle of blood cell counting plates. Take a classic blood cell counting plate as an example, only a portion of a limited volume (random sampling) in a counting pool is imaged and counted, and the imaging and counting of the entire counting pool cannot be realized. An uneven distribution of the cells in the counting region may create an inherent distribution error, which is significantly increased when the cell concentration is low, thereby greatly reducing the counting accuracy. Therefore, there is an urgent need for a counting plate that enables a full sample volume imaging to improve the counting accuracy and enable a larger countable range.

### SUMMARY

One of the embodiments of the present disclosure provides a counting assembly including a main body. The main body may be provided with a concave sample tank. A bottom end of the sample tank may be plane. A cross-sectional area of the sample tank may gradually increase from the bottom end to a top end of the sample tank.

In some embodiments, the sample tank may have a shape of an inverted frustum of a cone, an inverted frustum of an elliptic cone, or an inverted frustum of a pyramid.

In some embodiments, an angle between an inner wall of the sample tank and a horizontal plane may be less than 90°.

In some embodiments, the main body may be a thin-walled structure.

In some embodiments, the thin-walled structure may have a uniform wall thickness.

In some embodiments, the thin-walled structure may have an opening in a lower portion of the main body.

In some embodiments, the thin-walled structure may include a hollow enclosed structure or a solid structure. The hollow enclosed structure may generally refer to a hollow between an outer wall of the main body and a sidewall of the sample tank. The solid structure may generally refer to a solid filling between the outer wall of the main body and the sidewall of the sample tank.

In some embodiments, the counting assembly may further include a cover plate. An upper surface of the main body may be in contact with a lower surface of the cover plate, and the cover plate may be provided with one or more sample filling holes and one or more air venting holes.

In some embodiments, the projections of the one or more sample filling holes and the one or more air venting holes on the horizontal plane may have no overlap with a projection of a bottom surface of the sample tank on the horizontal plane.

In some embodiments, an area of the lower surface of the cover plate may be greater than an area of the upper surface of the main body, so that the cover plate is able to completely cover the main body when the cover plate is over the main body. In some embodiments, a shape of the lower surface of the cover plate may be similar to the shape of the upper surface of the main body. For example, in some embodiments, both the lower surface of the cover plate and the upper surface of the main body may be circular, square, rectangular, etc. In some embodiments, the shape of the lower surface of the cover plate may be different from the shape of the upper surface of the main body. For example, the lower surface of the cover plate may be square, while the upper surface of the main body may be circular.

In some embodiments, shapes of the one or more air venting holes and/or the one or more sample filling holes may include a circular shape, an elliptical shape, a rectangular shape, a rhombic shape, or an irregular shape.

In some embodiments, a first protrusion may be provided on an upper side of an edge of the one or more sample filling holes and/or the one or more air venting holes. The first protrusion may face toward an opposite side of the main body for preventing a sample overflow caused by a slight overfilling of the sample and thus increasing a fault tolerance of an operation.

In some embodiments, a second protrusion may be provided on an underside of an edge of the one or more sample filling holes and/or the one or more air venting holes, and an outer side of the second protrusion is in contact with the inner wall of the sample tank.

In some embodiments, the bottom surface of the sample tank may be smaller than an imaging target surface of a camera, so as to ensure that a single imaging is able to fully cover an imaging view. Those skilled in the art may be aware of an area range of an imaging target surface of the camera.

In some embodiments, a diameter of the bottom surface of the sample tank may be in a range from 1.5 mm to 2.0 mm.

One of the embodiments of the present disclosure provides a counting device including one or more counting assemblies as described in any embodiment of the present disclosure.

In some embodiments, the counting device may include a cover plate and a carrier plate, the carrier plate being provided with at least one hole matching with the counting assembly, a diameter of the at least one hole being in a range between an outer diameter of the main body and a diameter of the cover plate, so that the main body is able to be inserted into the at least one hole and the cover plate may be stuck outside the at least one hole.

In some embodiments, a count of the at least one hole on the carrier plate may be 24, 48, or 96, and each of the at least one hole may be attached to the counting assembly.

In some embodiments, the carrier plate may be integrally molded or detachably connected with the counting assembly. The detachable connection may be a connection that relies on a friction between the outer wall of the body and the inner wall of the at least one hole to enable the main body to snap into the at least one hole. In some embodiments, when an outer contour of the main body is of different shapes (e.g. rectangular), the at least one hole may also be provided in corresponding shapes.

In some embodiments, a supporting edge may be provided on a side of the carrier plate, a height of the supporting edge extending towards a bottom side of the counting assembly may be greater than a height of the counting assembly.

One of the embodiments of the present disclosure provides an application of a counting assembly and/or a counting device in the field of particle counting analysis. The counting assembly may be a counting assembly as described in any of the embodiments of the present disclosure. The counting device may be a counting device as described in any of the embodiments of the present disclosure. With the application, imaging and counting of cells, bacteria, fungi, or other particles in a full volume (e.g., 5 μL, 20 μL, 100 μL, 200 μL, 300 μL, 400 μL, 500 μL) of the sample in the sample tank may be realized. The application may be particularly suitable for counting and testing cells, bacteria, fungi, or other particles in samples of low concentration (as low as 0 particles/ml).

### BRIEF DESCRIPTION OF THE DRAWINGS

The present disclosure will be further illustrated by way of exemplary embodiments, which will be described in detail by means of the accompanying drawings. These embodiments are not limiting, and in these embodiments, the same numbering indicates the same structure, wherein:

FIG. 1 is a schematic diagram illustrating a structure of a counting assembly according to some embodiments of the present disclosure;

FIG. 2 is a schematic diagram illustrating a top view of a counting assembly according to some embodiments of the present disclosure;

FIG. 3 is a schematic diagram illustrating a structure of a carrier plate of the counting assembly according to some embodiments of the present disclosure;

FIG. 4 is a schematic diagram illustrating a structure of a counting device according to some embodiments of the present disclosure;

FIG. 5 is a schematic diagram illustrating a structure of a counting device according to some other embodiments of the present disclosure; and

FIG. 6 is a schematic diagram illustrating a sectional view of the counting device according to some other embodiments of the present disclosure.

Descriptions of the accompanying drawings markings: 100—counting assembly, 110—main body, 120—cover plate, 121—sample filling hole, 122—air venting hole, 200—carrier plate, 210—hole, 220—supporting edge.

### DETAILED DESCRIPTION

In order to more clearly illustrate the technical solutions of the embodiments of the present disclosure, the accompanying drawings to be used in the description of the embodiments will be briefly described below. Obviously, the accompanying drawings in the following description are only some examples or embodiments of the present disclosure, and it is possible for those skilled in the art to apply the present disclosure to other similar scenarios in accordance with these accompanying drawings without creative labor. Unless obviously obtained from the context or the context illustrates otherwise, the same numeral in the drawings refers to the same structure or operation. As indicated in the present disclosure and in the claims, unless the context clearly suggests an exception, the words “one,” “a,” “a kind of,” and/or “the” are not intended to be specifically to the singular, but may also include the plural.

Embodiments of the present disclosure relate to a counting assembly. The counting assembly may be capable of enriching cells, bacteria, fungi, or other particles in a sample in a sample tank at a bottom, and by directly observe or imaging the bottom plane, imaging and counting of the cells, the bacteria, the fungi, or the other particles in a full volume (e.g., 5 μL, 20 μL, 100 μL, 200 μL, 300 μL, 400 μL, 500 μL) of a sample in a sample tank may be realized. The counting assembly may be particularly suitable for counting and testing the cells, the bacteria, the fungi, or the other particles in samples of low concentration.

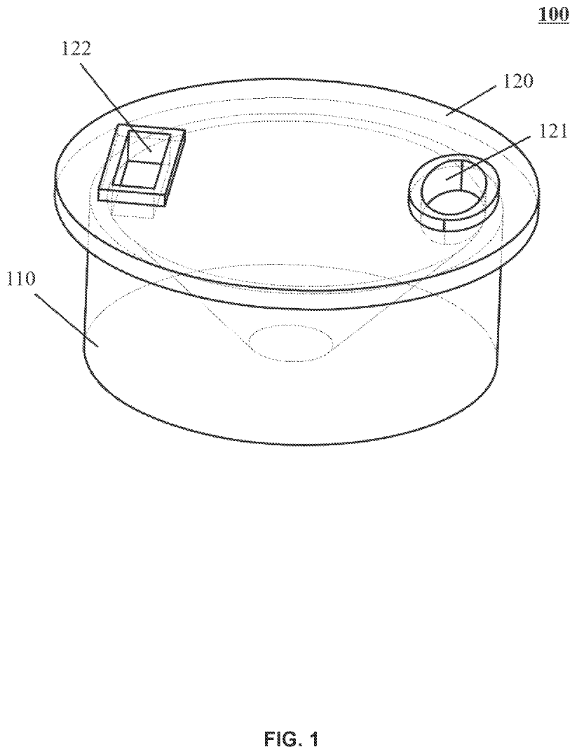

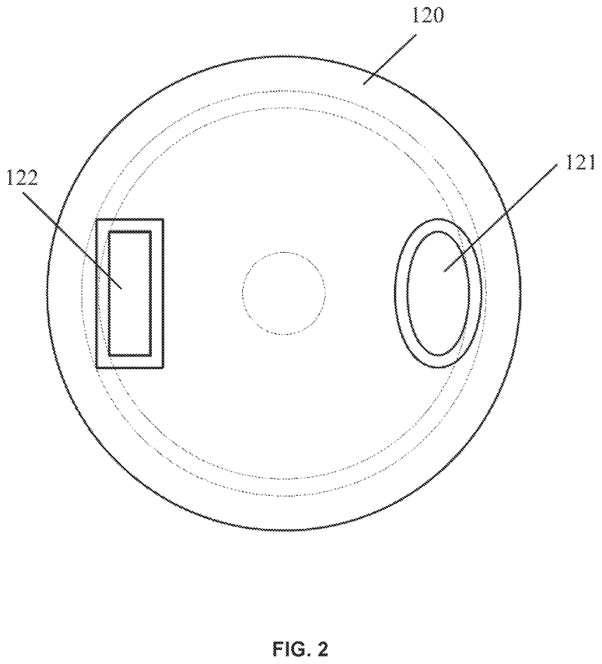

FIG. 1 is a schematic diagram illustrating a structure of a counting assembly according to some embodiments of the present disclosure, and FIG. 2 is a schematic diagram illustrating a top view of a counting assembly according to some embodiments of the present disclosure. The counting assembly involved in the embodiments of the present disclosure may be described in detail below in combining FIG. 1 and FIG. 2. It should be noted that the following embodiments are used only to explain the present disclosure and do not constitute a limitation of the present disclosure.

As shown in FIGS. 1 and 2, a counting assembly 100 may include a main body 110 and a cover plate 120. The main body 110 may be provided with a concave sample tank (as shown by a dotted line within the main body 110 in FIG. 1). A bottom end of the sample tank may be planar, and the sample tank may have a cross-sectional area that gradually increases from the bottom end to a top end. By forming a structure in which a top cross-section is greater than a bottom cross-section and a side transits evenly and gently, a greater volume of liquid may be accommodated in the sample tank. By centrifuging the main body with the sample, objects to be counted in the sample (e.g., cells, bacteria, fungi, or other particles, etc.) may be enriched on a bottom surface of the sample tank due to the centrifugal force. In some embodiments, the objects to be counted in the sample may also be enriched on the bottom surface of the sample tank by means of standing, magnetic attraction, etc. After the enrichment of the objects to be counted is completed, the objects may be observed and counted through a microscope, or may be counted and analyzed after taking images with an automatic counter. When an imaging view of a camera of the automatic counter is able to completely cover a bottom plane of the main body 110, the imaging and counting analysis may be realized by a single imaging. When the imaging view of the camera of the automatic counter is not able to completely cover the bottom plane of the main body 110, the imaging and counting analysis may be performed by taking a plurality of images and then stitching the images together. In some embodiments, a bottom surface of the sample tank may be a circle, and a diameter of the circle may be 1.5-2.0 mm, which is smaller than an actual imaging target surface of the camera, so as to ensure that the imaging view of a single imaging may be completely cover the bottom surface of the sample tank. In some embodiments, those skilled in the art may select a centrifuge based on a size of the main body 110, or may determine the size of the body 110 based on a specification of an existing centrifuge.

In some embodiments, the sample tank may have a shape of an inverted frustum of a cone. In other embodiments, the sample tank may have a shape of an inverted frustum of an elliptic cone. In some other embodiments, the sample tank may have a shape of an inverted frustum of a pyramid. In some embodiments, as shown in FIGS. 1 and 2, the sample tank may have the shape of an inverted frustum of the cone (e.g., a top inner diameter of 9 mm, a depth of 3.7 mm, and a bottom inner diameter of 2 mm). The sample may be located inside the inverted frustum of the cone after accommodated in the sample tank. After centrifugation, the object to be tested in the sample may be enriched in a bottom surface of the inverted frustum of the cone without accumulating on a sidewall. By disposing the sample tank in a regular shape (e.g., the inverted frustum of the cone), the sample tank may be made easy to process and manufacture. In some embodiments, the sample tank may be of other shapes. For example, in some embodiments, the sample tank may be any shape that satisfies a requirement that “the bottom end is flat, and the cross-sectional area gradually increases from the bottom end to the top end”.

In some embodiments, an angle between the inner wall of the sample tank and a horizontal plane may be 45°-60° (e.g., 45°, 50°, 60°, etc.), and the inwardly tilted sidewall may be favorable for the tested object to sink to the bottom, so as to be enriched on the bottom surface of the sample tank. In some embodiments, the angle between the inner wall of the sample tank and the horizontal plane may not be too small, because if the angle is too small, it may be difficult for the object to be tested to be fully enriched on the bottom of the sample tank. In some embodiments, the angle between the inner wall of the sample tank and the horizontal plane may not be too great (e.g., close to 90°), because if the angle is too great, the inner wall may cast a shadow when imaging and counting.

In some embodiments, the main body 110 may have a thin-walled structure to facilitate manufacturing (e.g., not easily deformed during the production) and to save manufacturing costs. In some embodiments, the thin-walled structure may have a uniform wall thickness. In some embodiments, the thin-walled structure may have an opening in a lower portion of the main body 110. In some embodiments, the main body 110 may be a hollow thin-walled closed structure with a hollow between the outer wall of the main body 110 and the sidewall of the sample tank. In other embodiments, the main body 110 may also be a solid structure, i.e., there is a solid fill between the outer wall of the main body 110 and the sidewall of the sample tank.

In some embodiments, the counting assembly 100 may further include the cover plate 120, an upper surface of the main body 110 may be in contact with a lower surface of the cover plate 120, and the cover plate 120 may cover the main body 110 when the counting assembly is in use. In some embodiments, fixed connection between the main body 110 and the cover plate 120 may be realized by means of bonding, snap-fitting, or integral molding, or in some other embodiments, the main body 110 and the cover plate 120 may merely be in contact with each other but not fixedly connected. The cover plate 120 may be respectively provided with one or more air venting holes 122 for venting air and one or more sample filling holes 121 for sampling. The one or more air venting holes 122 and the one or more sample filling holes 121 may be of the same shape or of different shapes. The shapes of the one or more air venting holes 122 or the one or more sample filling holes 121 may be circular, elliptical, rectangular, rhombic, or irregularly shaped, etc.

In some embodiments, an area of the lower surface of the cover plate 120 may be greater than an area of the upper surface of the main body 110 to enable the cover plate 120 to completely cover the main body 110 when the cover plate 120 is over the main body 110. In some embodiments, the shape of the lower surface of the cover plate 120 may be similar to the shape of the upper surface of the main body 110. For example, the lower surface of the cover plate 120 and the upper surface of the main body 110 may both be circular, square, rectangular, etc. In some embodiments, the shape of the lower surface of the cover plate 120 may be different from the shape of the upper surface of the main body 110. For example, the lower surface of the cover plate 120 may be square, while the upper surface of the main body 110 may be circular. For example, in the embodiments shown in FIGS. 1-2, both the cover plate 120 and an outer contour of the main body 110 are cylindrical. In some embodiments, a diameter of the cover plate 120 may be greater than a diameter of the main body 110. For example, the cylindrical main body 110 may an outer diameter of 6 mm, 7 mm, 8 mm, 9 mm, or 10 mm, and the circular cover plate 120 may have a diameter of 11.5 mm or 12 mm.

In some embodiments, projections of the one or more sample filling holes 121 and the one or more air venting holes 122 on the horizontal plane may not overlap with a projection of the bottom surface of the sample tank on the horizontal plane. In this way, impacts of the contour structure of the one or more sample filling holes 121 or the one or more air venting holes 122 on the counting during the microscope observation or imaging photography may be avoided. For example, in one of the embodiments as shown in FIG. 2, the elliptical sample filling hole 121 (e.g., with an inner diameter of 3 mm on a long axis and 1.5 mm on a short axis) and the rectangular air venting hole 122 (e.g., with a length of 3 mm and a width of 1 mm) may be symmetrically disposed along a radial direction of the cover plate 120 at a proximal edge of the cover plate 120. The bottom plane of the sample tank may be disposed just below a center position of the cover plate 120. In some other embodiments, the one or more sample filling holes 121 and the one or more air venting holes 122 may also be provided at other proximal edge locations on the cover plate 120, and the bottom plane may also be provided at a location directly below the center location of the cover plate 120, such that the projections of the one or more sample filling holes 121 and the one or more air venting holes 122 in the horizontal plane do not overlap with the projection of the bottom surface of the sample tank in the horizontal plane.

In some embodiments, the edges of the one or more sample filling holes 121 and the one or more air venting holes 122 may face upward, and a first protrusion may be provided on one side, as shown in FIG. 1. The first protrusion may face toward an opposite side of the main body 110 for preventing a sample overflow caused by a slight overfilling of the sample and thus increasing the fault tolerance of operation. In some other embodiments, only the edge of the one or more sample filling holes 121 may face upward with a first protrusion on one side, or the edge of the one or more air venting hole 122 may face upward with the first protrusion on one side, so that a sample overflow may be prevented to a certain degree.

In some embodiments, the one or more sample filling holes 121 and the one or more air venting holes 122 may be provided with the edges facing downward and with a second protrusion on one side. The outer side of the second protrusion may be in contact with the inner wall of the sample tank. In some embodiments, the second projection may be used in a situation where the main body 110 and the cover plate 120 are in contact with each other without a fixed connection. When the cover plate 120 is covered, the second protrusion may play a role in limiting and jamming the cover plate 120, so that after the cover plate 120 is placed upon the main body 110, the cover plate 120 may not deflect. At the same time, it may be also ensured that the one or more sample filling holes 121 and the one or more air venting holes 122 are connected to the inside of the sample tank, so as to optimize the efficacy of sample filling and venting. In some embodiments, utilizing the second protrusion, the main body 110 and the cover plate 120 may be more conveniently put into place during installation. In some other embodiments, only the edge of the one or more sample filling holes 121 may face downward, and the second projection may be provided on one side, or only the edge of the air venting hole 122 may face downward, and the second projection may be provided on one side. In some embodiments, an outer side of the second projection may be in contact with the inner wall of the sample tank, so as to cause a certain limiting effect on the cover plate 120.

Embodiments of the present disclosure relates to a counting device. The counting device may include one or more counting assemblies 100 of any of the above technical solutions. With the application of the one or more counting assemblies 100, the cells, the bacteria, the fungi, or the other particles, etc. in the sample may be enriched at the bottom of the sample tank. In this way, the imaging and counting of the cells, the bacteria, the fungi, or the other particles, etc. in the full volume of the sample in the sample tank may be realized. The counting device may be particularly suitable for imaging and counting of cells, bacteria, fungi, or other particles, etc. in a low concentration sample.

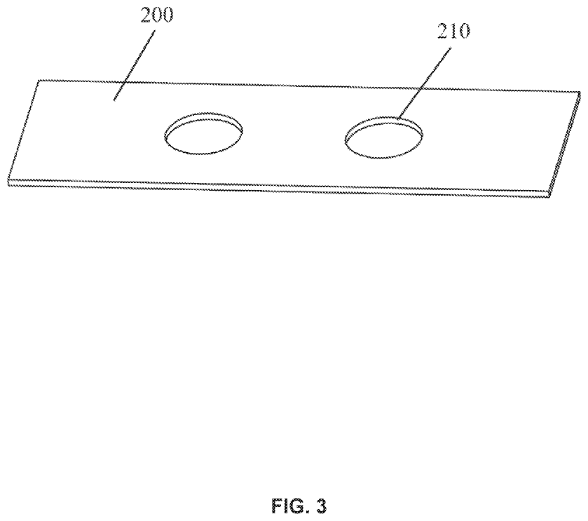

FIG. 3 is a schematic diagram illustrating a structure of a carrier plate of the counting assembly according to some embodiments of the present disclosure. As shown in FIG. 3, in some embodiments, a counting device may further include a carrier plate 200. The carrier plate 200 may be opened with at least one holes 210 matching one or more counting assemblies 100, so that the main body 110 is able to be inserted into the hole 210, and the cover plate 120 is able to be stuck outside the hole 210. For example, the holes 210 may have a diameter of 11 mm, the cylindrical main body 110 may have an outer diameter of 10.5 mm, and the circular cover plate 120 may have a diameter of 12 mm. For another example, the hole 210 may have a diameter of 10.5 mm, the outer diameter of the cylindrical main body 110 may be 9.83 mm, and the diameter of the cover plate 120 may be 11.5 mm.

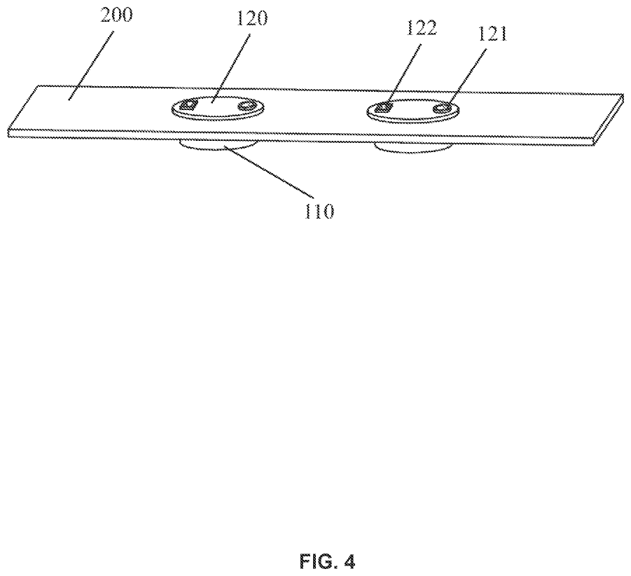

FIG. 4 is a schematic diagram illustrating a structure of a counting device according to some embodiments of the present disclosure. As shown in FIG. 4, in some embodiments, the carrier plate 200 may have a small number of holes 210 (e.g., there may be 2, 3, 4, 5 holes 210, etc.), and the main body 110 may be movably inserted into the holes 210 and detachably connected to the carrier plate 200. In some embodiments, the detachable connection may be realized by disposing the hole 210 between the main body 110 and the cover plate 120, wherein the main body 110 is fixedly connected to the cover plate 120, and the diameter of the cover plate 120 is greater than the outer diameter of the main body 110 (e.g., the diameter of the hole 210 may be 10.5 mm, the maximum outer diameter of the main body 110 may be 9.83 mm, and the diameter of the cover plate 120 may be 11.5 mm). The main body 110 may be inserted into the hole 210 and erected on the carrier plate 200 through the cover plate 120. In some embodiments, the detachable connection may be realized by a friction between the outer wall of the main body 110 and the inner wall of the hole 210, wherein the main body 110 is stuck into the hole 210. In some embodiments, when an outer contour of the main body 110 is of one shape (e.g., rectangular), the hole 210 may also be disposed to a corresponding shape.

In this embodiment, the one or more counting assemblies 100 may be separated from the carrier plate 200, and a size of the individual counting assembly 100 may be matched to an existing centrifugal device, so that the counting assembly may be individually centrifuged in the centrifugal device, enabling the object to be detected to sink to the bottom. Then the object to be detected may be placed into the hole 210 of the carrier plate 200, and then counted and analyzed using a microscope or an automated counter.

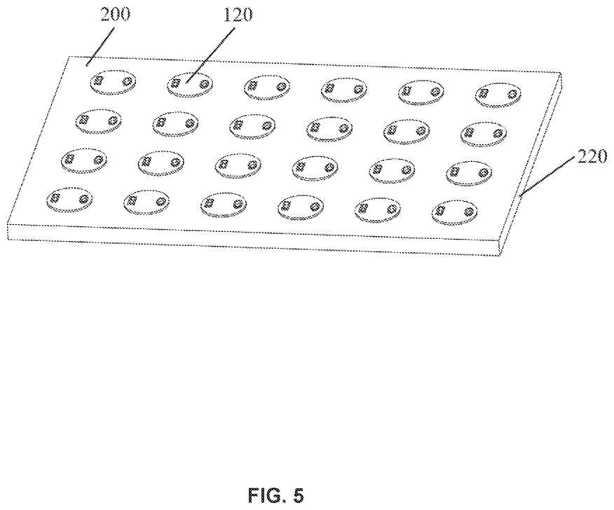

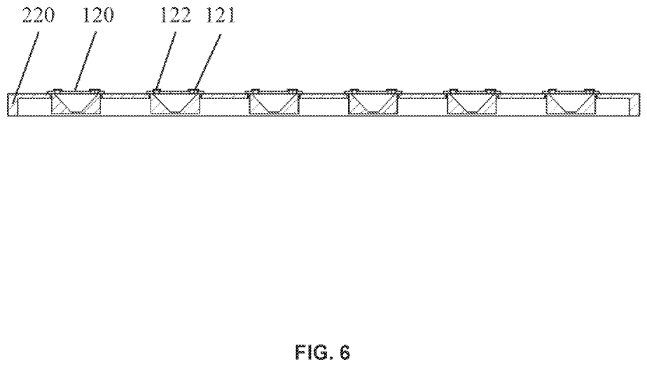

FIG. 5 is a schematic diagram illustrating a structure of a counting device according to some other embodiments of the present disclosure; and FIG. 6 is a schematic diagram illustrating a sectional view of the counting device according to some other embodiments of the present disclosure. As shown in FIGS. 5 and 6, in some embodiments, a plurality of holes 210 (e.g., 24 holes, 48 holes, 96 holes) may be provided on the carrier plate 200, with a counting assembly 100 fixedly mounted in each of the holes 210 to form a high-throughput counting device. The fixed connection may be bonding. For example, the bonding may be UV adhesive bonding or glue injection liquid bonding. For example, a lower surface edge of the cover plate 120 of the counting assembly 100 may be bonded to the carrier plate 200 by adhesive liquid bonding. In some embodiments, the carrier plate 200 and the counting assembly 100 may be integrally molded to form an integrated counting device with the same size and specification as an existing carrier plate of 24 holes, 48 holes, or 96 holes. In some embodiments, the counting device may be matched to a centrifugation device so that the counting device may be placed integrally into the centrifugation device for centrifugation, allowing an object to be detected to sink to the bottom. Then a count and analysis may be performed using an automated counter.

In some embodiments, a supporting edge may be provided on an edge of the carrier plate 200, a height of the supporting edge 220 extending towards a bottom side of the counting assembly 100 may be greater than a height of the counting assembly. The supporting edge 220 may be provided on any opposite sides of the carrier plate 200, or may be provided on all four sides of the carrier plate 200. The bottom of the supporting edge 220 may be in direct contact with a tabletop, a countertop, etc. when the carrier plate 200 is placed, so as to avoid the bottom of the counting assembly 100 from abraded by the direct contact with the tabletop, the countertop, etc. and affect the counting analysis.

In some embodiments, a material of the counting device (e.g., the main body, the cover plate, and/or the carrier plate) may be the material that is highly transparent and chemically resistant. For example, the material may include polypropylene, polystyrene, etc.

Possible beneficial effects of the embodiments of the present disclosure may include, but not limited to: (1) the counting device may be washable and reusable, or may be disposable, and when used as a disposable device, it may be easy to operate and may not require mounting and cleaning; (2) the imaging and counting of the full volume of particles or cells, etc. of the sample in the sample tank may be realized by imaging the sample for at least once, which makes the testing fast and efficient, thereby solving and eliminating the counting error generated by the distribution of the particles or cells and/or a limited sampling of the particles or cells, etc.; (3) the sample volume of a single test may be up to 500 μL, and a lower limit range of the sample concentration test may be greatly expanded. The lowest sample volume may be down to 0/ml, which is especially suitable for counting of the low-concentration sample. It is to be noted that in the present disclosure, the sizes of the counting assembly and the counting device may be adjusted according to application requirements, so that the counting assembly and the counting device may be applied to microparticle counting, precise counting, and fluorescence analysis of various types of cells, as well as applied in the fields of blood cell counting in medical clinical analysis, and analysis of formed components of urine. It may be noted that different embodiments may produce different beneficial effects, and in different embodiments, the beneficial effects that may be produced may be any one or a combination of any one or more of the above, or any other beneficial effect that is able to be obtained.

Obviously, to those skilled in the art, the above detailed disclosure may be intended only as an example and may not constitute a limitation of the present disclosure. Although not expressly stated herein, those skilled in the art may make various modifications, improvements, and amendments to the present disclosure. Such modifications, improvements, and amendments are suggested in the present disclosure, so such modifications, improvements, and amendments remain within the spirit and scope of the exemplary embodiments of the present disclosure.

Also, the present disclosure uses specific words to describe the embodiments of the present disclosure. For example, “an embodiment”, “one embodiment”, and/or “some embodiments” are meant to refer to a certain feature, structure, or characteristic associated with at least one embodiment of the present disclosure. Accordingly, it should be emphasized and noted that “an embodiment” or “one embodiment” or “an alternative embodiment” mentioned two or more times in different places in the present disclosure do not necessarily refer to the same embodiment. Furthermore, certain features, structures, or characteristics in one or more embodiments of the present disclosure may be suitably combined.

In addition, it should be noted that, in order to simplify the presentation of the disclosure of the present disclosure, and thus to aid in the understanding of one or more embodiments of the present disclosure, the foregoing descriptions of embodiments of the present disclosure sometimes combine a variety of features into a single embodiment, accompanying drawings, or a description thereof. However, this mode of disclosure does not imply that the objects of the present disclosure require more features than those mentioned in the claims. Rather, the claimed subject matter may lie in less than all features of a single foregoing disclosed embodiment.

Some embodiments use numbers describing the count of components, attributes, and it may be understood that such numbers used in the description of the embodiments are modified in some examples by the modifiers “about”, “approximately”, or “substantially”. Unless otherwise stated, “about”, “approximately”, or “generally” indicates that a variation of ±20% is permitted in the described numbers. Accordingly, in some embodiments, the numerical parameters used in the present disclosure and claims are approximations, which change depending on the desired characteristics of the individual embodiment. In some embodiments, the numerical parameters should consider a specified number of valid digits and employ general bit retention. Although the numerical domains and parameters used to confirm the breadth of their ranges in some embodiments of the present disclosure are approximations, in specific embodiments such values are set as accurately as practicable.

Finally, it should be understood that the embodiments described in the present disclosure are only used to illustrate the principles of the embodiments of the present disclosure. Other variations may also fall within the scope of the present disclosure. Thus, as examples and not limitations, alternative configurations of the embodiments of the present disclosure may be considered consistent with the teachings of the present disclosure. Accordingly, the embodiments of the present disclosure are not limited to the embodiments expressly presented and described herein.

1. A counting assembly comprising a main body, wherein
the main body is provided with a concave sample tank, a bottom end of the sample tank is plane, and a cross-sectional area of the sample tank gradually increases from the bottom end to a top end of the sample tank.

2. The counting assembly of claim 1, wherein the sample tank has a shape of an inverted frustum of a cone, an inverted frustum of an elliptic cone, or an inverted frustum of a pyramid.

3. The counting assembly of claim 1, wherein an angle between an inner wall of the sample tank and a horizontal plane is less than 90°.

4. The counting assembly of claim 3, wherein the angle between the inner wall of the sample tank and the horizontal plane is 45°-60°.

5. The counting assembly of claim 1, wherein the main body has a thin-walled structure, and the thin-walled structure has a uniform wall thickness.

6. (canceled)

7. The counting assembly of claim 5, wherein the thin-walled structure has an opening in a lower portion of the main body.

8. The counting assembly of claim 5, wherein the thin-walled structure includes a hollow enclosed structure, or a solid structure.

9. The counting assembly of claim 1, further comprising:
a cover plate,
an upper surface of the main body is in contact with a lower surface of the cover plate,
wherein the cover plate is provided with one or more sample filling holes and one or more air venting holes.

10. The counting assembly of claim 9, wherein projections of the one or more sample filling holes and the one or more air venting holes on a horizontal plane have no overlap with a projection of the bottom end of the sample tank on the horizontal plane.

11. The counting assembly of claim 9, wherein an area of the lower surface of the cover plate is greater than an area of the upper surface of the main body.

12. The counting assembly of claim 9, wherein the one or more air venting holes and/or the one or more sample filling holes are of a circular shape, an elliptical shape, a rectangular shape, a rhombic shape, or an irregular shape.

13. The counting assembly of claim 9, wherein a first protrusion is provided on an upper side of an edge of the one or more sample filling holes and/or the one or more air venting holes.

14. The counting assembly of claim 9, wherein a second protrusion is provided on an underside of an edge of the one or more sample filling holes and/or the one or more air venting holes, and an outer side of the second protrusion is in contact with an inner wall of the sample tank.

15. The counting assembly of claim 1, wherein the bottom surface of the sample tank is smaller than an imaging target surface of a camera.

16. The counting assembly of claim 15, wherein a diameter of the bottom surface of the sample tank is in a range from 1.5 mm to 2.0 mm.

17. A counting device comprising a counting assembly including a main body, wherein
the main body is provided with a concave sample tank, a bottom end of the sample tank is plane, and a cross-sectional area of the sample tank gradually increases from the bottom end to a top end of the sample tank.

18. The counting device of claim 17, wherein the counting device includes a cover plate and a carrier plate, the carrier plate being provided with at least one hole matching with the counting assembly, wherein a diameter of the at least one hole is in a range between an outer diameter of the main body and a diameter of the cover plate.

19. The counting device of claim 18, wherein a count of the at least one hole on the carrier plate is 24, 48, or 96, and each of the at least one hole is attached to each of the one or more counting assemblies.

20. The counting device of claim 19, wherein the carrier plate is integrally molded or detachably connected with the one or more counting assemblies.

21. The counting device of claim 19, wherein one or more supporting edges is provided on a side of the carrier plate, and a height of the one or more supporting edges extending towards a bottom side of the counting assembly is greater than a height of the counting assembly.

22-23. (canceled)
